(19) United States
(12) Patent Application Publication
(10) Pub. No.: US 2026/0144236 A1
(43) Pub. Date: May 28, 2026

(54) Systems and Methods for Remote Monitoring and Well-Being Management of Horses

(71) Applicant: John Fischbeck, SAN JUAN CAPISTRANO, CA (US)

(72) Inventor: John Fischbeck, SAN JUAN CAPISTRANO, CA (US)

(21) Appl. No.: 18/955,986

(22) Filed: Nov. 22, 2024

(51) Int. Cl. A01K 29/00; A01K 11/00; A61B 5/00; A61B 5/0205; A61B 5/024; A61B 5/11; A61B 5/16; G06V 10/82; G06V 40/10

(52) U.S. Cl. CPC A01K 29/005; A01K 11/008; A61B 5/0022; A61B 5/02055; A61B 5/1112; A61B 5/1118; A61B 5/112; A61B 5/165; A61B 5/4266; A61B 5/6829; A61B 5/746; A61B 5/7475; A61B 5/02438; A61B 2503/40; A61B 2560/0223; G06V 10/82; G06V 40/10

(57) ABSTRACT

The invention provides a system for monitoring the health, behavior, and safety of horses using a wearable device and an AI-enabled camera. The wearable device, affixed to the horse's pastern, measures physiological and motion data through multi-modal sensors, while the AI camera captures high-resolution facial images to assess mood states such as stress, happiness, discomfort, and pain. Data is processed locally with artificial intelligence algorithms and transmitted via WiFi, LoRa, GSM, or Bluetooth to a cloud-based platform. The platform provides real-time alerts, tracks historical trends, and enables data sharing among caregivers, veterinarians, and researchers. The system includes geofencing for safety, redundant communication protocols for reliability, and modular designs for adaptability across environments. By integrating physical, mental, and environmental monitoring, the invention delivers actionable insights to support proactive care, ensuring the health, safety, and welfare of horses.

### PRIOR ART

Here are brief descriptions of the prior art documents provided:

U.S. Pat. No. 8,412,315 (Analysis of Heart Rate Variability) (U.S. Pat. No. 8,412,315):
a. Focuses on analyzing heart rate variability (HRV) in animals to assess their autonomic nervous system and overall health. The system collects heart rate data, performs HRV analysis, and provides insights into physiological states and potential health conditions. It emphasizes the use of frequency and time domain analysis for animal monitoring.

U.S. Pat. No. 9,355,307 (Real-Time Performance Assessment) (U.S. Pat. No. 9,355,307):
a. Describes a method and apparatus for real-time monitoring of animal performance. This includes sensors for collecting physiological, biochemical, and kinematic data. The system transmits data to a secondary device for live or recorded analysis. Applications include assessing metabolic and physical states during training or competitive activities.

U.S. Pat. No. 10,561,365 (Monitoring Equine Condition) (U.S. Pat. No. 10,561,365):
a. A system designed specifically for monitoring equine behavior and condition. It includes sensors to track parameters over time, establish behavioral baselines, and detect abnormalities. Data is stored in a database and used for generating insights into equine health and well-being.

US20170055496A1 (Animal Health Sensor System) (US20170055496A1):
a. Focuses on a system integrating sensors with wireless communication for monitoring physiological properties of animals. Sensors are incorporated into patches affixed to the animal's body, such as the tail or hoof, for collecting temperature and movement data. The system transmits data to a server for analysis and remote access.

US20200093101A1 (State Detection System for Animals) (US20200093101A1):
a. Describes a system for detecting the behavioral state of animals using accelerometers to monitor movements. It identifies states like feeding, resting, or ruminating based on predefined thresholds and analyzes data to provide insights into animal health and behavior.

WO2016138571A1 (Integrated Animal Monitoring) (WO2016138571A1):
a. Presents a comprehensive animal monitoring system that integrates multiple sensors to track physiological and environmental parameters. The system uses machine learning to analyze data, identify patterns, and predict health-related outcomes, offering applications in livestock and companion animal care.

### BACKGROUND OF THE INVENTION

Horses play an essential role in industries such as racing, equestrian sports, recreation, agriculture, and companionship. Their health, behavior, and safety are of paramount importance, requiring constant attention to ensure their well-being and performance. Despite advancements in monitoring technologies, current solutions remain fragmented and fail to provide a comprehensive view of a horse's condition, encompassing its physical, mental, and environmental health.

### Challenges in Current Systems

### Physical Health Monitoring

a. Existing tools, such as heart rate monitors or accelerometers, focus on isolated parameters like cardiovascular activity or gait analysis. These devices lack the capability to integrate physiological data with behavioral and environmental insights, which are critical for identifying underlying health issues.

### Mental and Emotional Health Assessment

a. The mental and emotional states of horses, including stress, happiness, fatigue, and discomfort, are often overlooked. While facial expressions such as ear position, eye openness, and nostril flare provide valuable indicators of mood, current systems lack the advanced imaging and AI technologies necessary to capture and analyze these subtle cues.

### Environmental Health Factors

a. Environmental conditions such as temperature, humidity, and air quality significantly impact a horse's natural health and performance. However, existing environmental monitoring systems operate independently, without correlating these factors with the horse's physiological and behavioral data.

### Safety Concerns

a. Geolocation and geofencing tools are widely used to monitor a horse's location during transport or while grazing in open pastures. These systems, however, are disconnected from health and behavior monitoring, limiting their ability to provide holistic safety solutions.

### Fragmentation and Limited Insights

a. Current technologies fail to unify data streams from wearables, environmental sensors, and behavioral tools into a single, cohesive platform. As a result, caregivers lack the ability to detect patterns, predict risks, or respond proactively to emerging health or safety concerns.

### Communication and Connectivity Limitations

a. Many systems rely on a single communication protocol, such as WiFi or GSM, making them unreliable in remote or large-scale environments. The lack of redundancy and flexibility in communication pathways further limits the effectiveness of these systems.

### Opportunities for Technological Integration

Advancements in wearable technology, artificial intelligence (AI), and Internet of Things (IoT) communication provide new opportunities for overcoming these challenges. AI-powered systems, such as convolutional neural networks (CNNs), enable the analysis of complex data, including facial expressions and motion patterns, to identify health risks and emotional states. When combined with wearable devices and robust communication protocols, these technologies create a platform for comprehensive, real-time monitoring of a horse's overall well-being.

### Purpose of the Invention

The present invention aims to bridge the gaps in traditional equine care by providing an integrated system that unifies physical, mental, and environmental monitoring. By combining wearable devices with multi-modal sensors, AI-enabled imaging for facial recognition, and adaptive communication protocols, the invention delivers real-time insights into a horse's health, behavior, and safety. This system recreates a “day in the life” of the horse, offering caregivers a detailed view of its well-being while enabling early detection of health risks and behavioral anomalies.

In addition to health monitoring, the invention enhances safety through geofencing, real-time alerts, and the ability to reconstruct historical data. These features are supported by a cloud-based platform that correlates data streams, identifies trends, and provides actionable insights for caregivers, veterinarians, and researchers.

This invention represents a significant advancement in equine monitoring technology, addressing the limitations of existing systems and enabling proactive, data-driven care to improve the health, safety, and welfare of horses across a variety of environments and applications.

### SUMMARY OF THE INVENTION

The present invention provides an integrated system, method, and device for real-time monitoring, analysis, and management of equine health, behavior, and safety. By combining wearable technology, AI-powered imaging, advanced sensors, and adaptive communication protocols, the invention delivers actionable insights into a horse's physical, mental, and environmental well-being. This unified approach addresses the limitations of traditional systems by correlating multiple data streams for a holistic understanding of the horse's condition.

A wearable device, securely affixed to the horse's pastern, serves as the primary data collection unit. It features multi-modal sensors for tracking physiological metrics, such as heart rate, temperature, perspiration, and motion patterns. The wearable device operates alongside an AI-enabled camera, which captures and analyzes high-resolution facial images to detect mood states, including stress, happiness, fatigue, and pain. The data from these two components is combined to provide a comprehensive “day in the life” view of the horse, enabling early detection of health risks and behavioral anomalies.

To ensure seamless and reliable data transmission, the invention incorporates a robust communication architecture, including WiFi, LoRa, GSM, and direct cellular transport. The system operates in diverse environments, from remote pastures to controlled stable settings, with flexibility for local or cloud-based processing. A cloud platform serves as the central repository for data storage, advanced analytics, and visualization. It provides real-time alerts, tracks historical trends, and supports collaboration among caregivers, veterinarians, trainers, and researchers.

Designed for adaptability, the system includes modular and scalable components that allow for customization based on the horse's breed, size, and environment. The wearable device, AI camera, and communication modules can be adjusted for specific use cases, such as training optimization, environmental health monitoring, or herd management.

This invention represents a significant advancement in equine care by integrating advanced technologies into a cohesive platform. It empowers stakeholders to make proactive, data-driven decisions, enhancing the health, safety, and welfare of horses across various applications.

### BRIEF DESCRIPTION OF DRAWINGS

The drawings illustrate various components and aspects of the invention, providing a visual understanding of its structure, functionality, and operational workflow. The reference numerals correspond to specific elements described in the invention.

FIG. 1: Depicts the anatomy of a horse's lower leg, including the cannon bone, fetlock joint, pastern, and hoof, highlighting the placement of the wearable device (6) on the pastern for secure and effective data collection.

FIG. 2: Illustrates a detailed breakout of the wearable device (6), showing the dual-reinforced pastern wrap, internal sensors for physiological and motion data, rechargeable battery (7), and communication modules, emphasizing its modular design and durability.

FIG. 3: Provides an exploded view of the wearable device (6), detailing internal components such as the microcontroller unit (24), LoRa, WiFi, and GSM modules (25, 26, 30), and sensors for tracking heart rate, temperature, and motion.

FIG. 4: Shows left and right views of the host server device (51), including features such as the SD card holder (40), USB-C port (41) for charging and data transfer, and the on/off switch (42), which facilitates local processing and communication.

FIG. 5: Offers an expanded breakout of the host server device (51), highlighting internal components, including the microcontroller unit (45), communication modules (44, 46, 47), and power management system, showcasing redundancy and reliability.

FIG. 6: Depicts the AI camera (52), including the camera module (51), electronics housing (52), and rechargeable battery (53). It captures high-resolution facial images for mood detection using convolutional neural networks.

FIG. 7: Provides a detailed view of the AI camera (52), featuring the microcontroller (54) with an SD card slot for local storage, WiFi and Bluetooth modules, housing screws (55), and battery connection system (56).

FIG. 8: Illustrates the CNN architecture of the wearable device (6), showing how it processes physiological and motion data to detect patterns indicative of health issues such as stress, fatigue, or discomfort.

FIG. 9: Highlights the key anatomical features of a horse's face, such as ears, eyes, nostrils, and mouth, analyzed by the AI camera (52) to identify mood states including stress, happiness, fatigue, or pain.

FIG. 10: Demonstrates the CNN architecture of the AI camera (52), detailing the processing of facial images to extract features, classify mood states, and generate probabilities for emotional conditions.

FIG. 11: Combines data from the wearable device (6) and AI camera (52), illustrating how the motion analysis CNN and facial recognition CNN work together to enhance prediction accuracy by correlating physical, behavioral, and environmental data.

FIG. 12: Depicts the communication architecture, showing interactions between the wearable device (6), AI camera (52), and host server device (51) via the local WiFi cloud (60) or directly to the internet cloud (65) using cellular transport (66). It also illustrates how the AI camera offloads data to the wearable device for transmission.

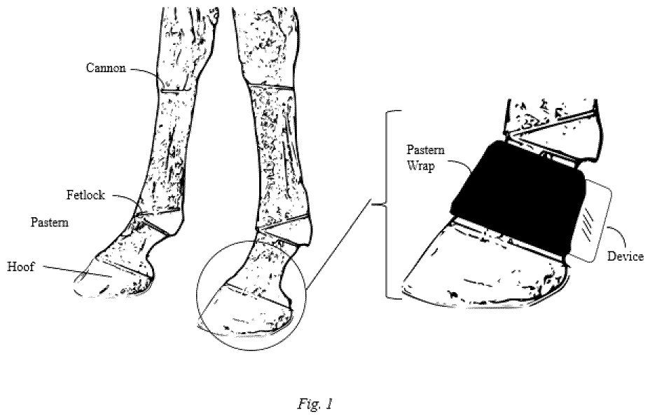

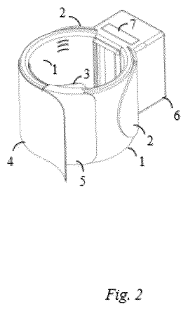

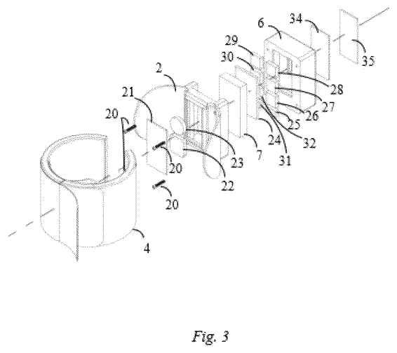

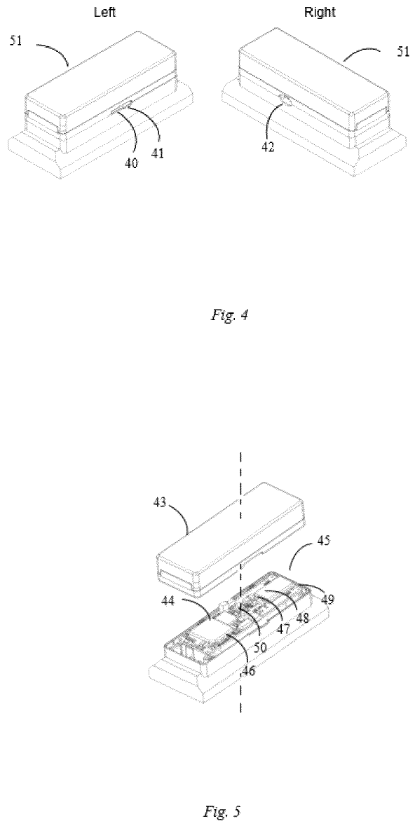

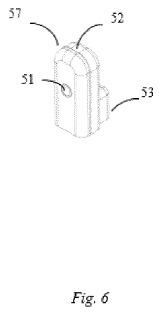

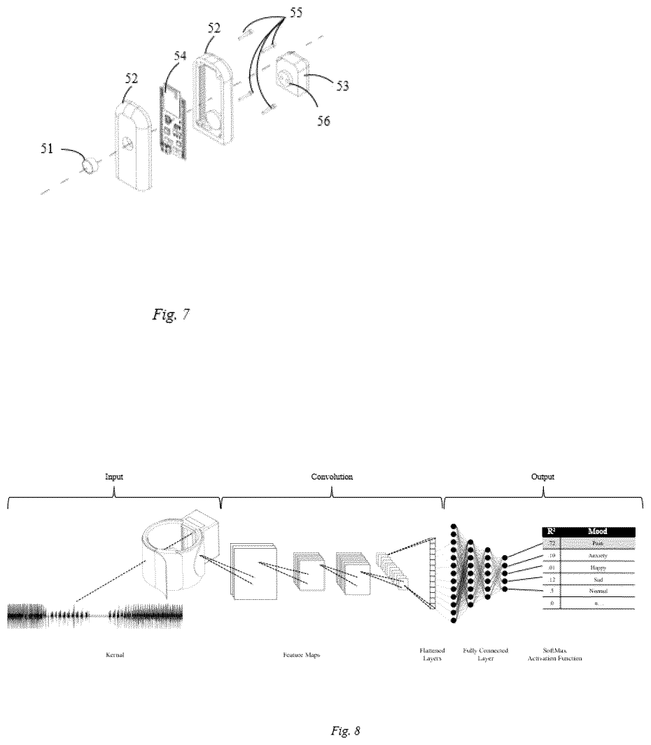

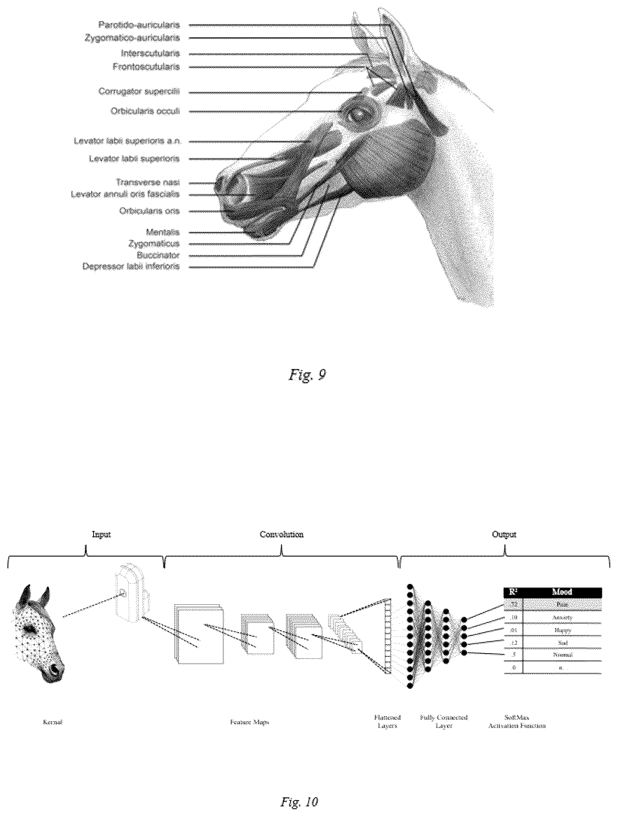

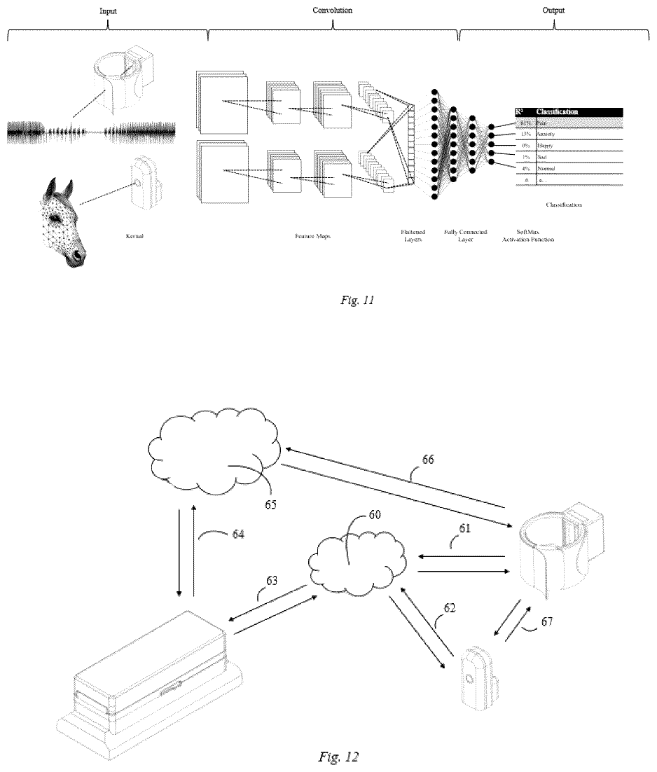

These figures collectively provide a comprehensive visual representation of the invention, clarifying its components and system architecture for equine health, behavior, and safety monitoring.

### DETAILED DESCRIPTION OF THE INVENTION

The present invention provides an integrated system, method, and device for monitoring, analyzing, and managing the health, behavior, and safety of horses. By leveraging wearable technology, AI-powered imaging, advanced sensors, and adaptive communication protocols, this invention offers real-time insights into a horse's physical, mental, and environmental well-being. This comprehensive solution addresses the limitations of existing systems by correlating multiple data streams into a unified and proactive platform for equine care.

### i. Wearable Device

The wearable device (6) is affixed to the horse's pastern using a dual-reinforced wrap for secure placement and optimal comfort. It serves as the primary data collection unit and incorporates the following components:

### Multi-Modal Sensors

a. Physiological Sensors: Measure heart rate, body temperature, and perspiration to assess physical health.
b. Motion Sensors: Utilize a 9-axis accelerometer and gyroscope to track activity patterns, gait, and movement irregularities.
c. Environmental Sensors: Monitor temperature, humidity, and air quality, correlating these factors with physiological and behavioral data.

### Power Module

a. A rechargeable battery (7) ensures long-term operation, with an optional solar charging feature for remote environments.

### Communication Module

a. Includes WiFi, LoRa, GSM, and Bluetooth protocols for seamless data transmission to the host server (51) or cloud platform (65).

### Microcontroller Unit (MCU)

a. Processes data locally and utilizes AI algorithms to detect health risks and anomalies before transmitting actionable insights.

### 2. AI Camera

The AI camera (52) complements the wearable device (6) by analyzing the horse's facial expressions to assess mental and emotional states. Its features include:

### High-Resolution Imaging

a. Captures key facial features such as ear position, eye openness, nostril flare, and mouth tension.

### Convolutional Neural Networks (CNNs)

a. Extract and classify facial features to detect mood states like stress, happiness, fatigue, or pain with high accuracy.

### Data Offloading

a. Transmits captured images to the wearable device (6) for cellular transport or directly to the cloud (65) via WiFi or GSM.

### Power and Housing

a. Equipped with a dedicated rechargeable battery (53) and a waterproof housing for reliable operation in diverse environments.

### 3. Communication Architecture

The invention features a robust communication system to ensure reliable data flow across various scenarios:

### Local WiFi Cloud (60)

a. Facilitates interaction between the wearable device (6), AI camera (52), and host server device (51).

### Direct Cellular Transport (66)

a. Provides an alternative pathway for data transmission when local WiFi infrastructure is unavailable.

### Host Server Device (51)

a. Acts as a local gateway, preprocessing data and forwarding it to the cloud for advanced analytics.

### Redundant Communication Protocols

a. Incorporates WiFi, LoRa, GSM, and Bluetooth for reliable connectivity, even in remote areas.

### 4. Cloud-Based Platform

The cloud platform (65) is the central hub for storing, processing, and visualizing data. It includes the following features:

### AI-Driven Analytics

a. Correlates data streams from the wearable device (6) and AI camera (52) to identify health risks, behavioral changes, and environmental stressors.

### Historical Data and Trend Analysis

a. Tracks long-term patterns to uncover recurring issues and deviations from normal behavior.

### Real-Time Alerts

a. Notifies caregivers of critical conditions such as geofence breaches, health anomalies, or significant behavioral shifts.

### Collaboration Tools

a. Allows secure data sharing among caregivers, veterinarians, trainers, and researchers for informed decision-making.

### 5. Use Cases

The invention applies to a variety of scenarios, including:

### Health Monitoring

a. Tracks physiological and behavioral metrics to detect early signs of illness or injury.

### Mental and Emotional Health

a. Analyzes facial expressions and behavioral data to assess stress, discomfort, or happiness.

### Environmental Management

a. Correlates environmental data with physiological and behavioral metrics to optimize living conditions.

### Safety

a. Uses geofencing and real-time alerts to monitor the horse's location and activity, reducing risks during transportation or in open pastures.

### Training Optimization

a. Combines motion and emotional data to refine training regimens and maximize performance.

### 6. Alternative Embodiments

The invention includes modular and scalable features to adapt to different environments and use cases:

### Wearable Variations

a. Customizable designs and sensor configurations accommodate various breeds, sizes, and conditions.

### Camera Placement

a. The AI camera (52) can be mounted on halters, stalls, or other structures as needed.

### Communication Scalability

a. Supports large-scale operations, such as monitoring herds in remote pastures.

### Advantages of the Invention

This invention offers significant improvements over existing systems by:

### Holistic Monitoring

a. Combines physiological, behavioral, and environmental data for a complete understanding of the horse's well-being.

### Real-Time Insights

a. Provides instant notifications and actionable recommendations based on AI-driven analytics.

### Reliability

a. Redundant communication protocols ensure uninterrupted data flow across diverse environments.

### Adaptability

a. Modular design allows customization for specific needs and applications.

### Proactive Care

a. Tracks historical data and identifies trends to support early intervention and preventive care.

This invention represents a transformative approach to equine monitoring, empowering caregivers with the tools to ensure the health, safety, and welfare of horses in any environment.

1. A system for monitoring the health, behavior, and safety of horses, comprising a wearable device configured to be affixed to the pastern of a horse, the wearable device including a plurality of sensors for measuring heart rate, temperature, perspiration, gait, activity patterns, and environmental factors; a microcontroller unit (MCU) for processing data locally using artificial intelligence algorithms to detect anomalies and behavioral patterns; a communication module supporting WiFi, LoRa, GSM, and Bluetooth; a rechargeable battery with optional solar charging for extended operation; an AI camera configured to capture and analyze high-resolution images of the horse's face for detecting mood states using convolutional neural networks (CNNs); a communication system for transmitting data from the wearable device and the AI camera to a host server or a cloud platform; and a cloud-based platform configured to store and analyze the data, generate real-time alerts, and provide a user interface for caregivers to access insights and historical trends.
The system of claim 1, wherein the AI camera further comprises a microcontroller for processing captured images, an SD card for local storage, and a communication module supporting WiFi and Bluetooth for data transfer.
The system of claim 1, further comprising a geofencing module integrated into the wearable device for monitoring the horse's location using GPS and generating alerts for boundary breaches.
The system of claim 1, further comprising a dynamic adaptability feature implemented through artificial intelligence algorithms to recalibrate the system for changes in device placement or newly identified behavioral patterns.
A method for monitoring the health and behavior of horses, comprising the steps of affixing a wearable device to the pastern of a horse to collect physiological, motion, and environmental data; capturing images of the horse's face using an AI camera for analysis of mood states through convolutional neural networks; processing the collected data locally on the wearable device and AI camera using artificial intelligence algorithms to detect patterns, anomalies, and risks; transmitting the processed data using WiFi, LoRa, GSM, or Bluetooth to a host server or cloud-based platform; generating real-time alerts based on the analyzed data; and providing access to real-time and historical data through a user interface for caregivers, veterinarians, and other stakeholders to make informed decisions.
